(19) United States
(12) Patent Application Publication
(10) Pub. No.: US 2004/0136994 A1
(43) Pub. Date: Jul. 15, 2004

(54) Methods of using CR3 and CR4 ligands for inhibiting interleukin-12 to treat autoimmune disease

(76) Inventors: Brian Kelsall, Washington, DC (US); Warren Strober, Bethesda, MD (US); Ivan Fuss, Bethesda, MD (US); Thomas Marth, Kensington, MD (US)

Correspondence Address: NATIONAL INSTITUTE OF HEALTH, C/O NEEDLE & ROSENBERG, P.C., SUITE 1000, 999 PEACHTREE STREET, ATLANTA, GA, 30303, US

(21) Appl. No.: 10/744,659

(22) Filed: Dec. 23, 2003

Related U.S. Application Data
(63) Continuation of application No. 09/196,867, filed on Nov. 20, 1998, now abandoned.
(60) Provisional application No. 60/066,238, filed on Nov. 20, 1997.

(51) Int. Cl. A61K 39/395

(52) U.S. Cl. 424/145.1

(57) ABSTRACT

The present invention provides a method of downregulating interleukin-12 production in a subject, comprising administering to the subject an interleukin-12 downregulating amount of a ligand of complement receptor 3 and/or complement receptor 4 effective in downregulating interleukin-12 production. Also provided is a method of reducing an interleukin-12-induced inflammatory response in a subject, comprising administering to the subject an amount of a ligand of complement receptor 3 and/or complement receptor 4 effective in reducing the interleukin-12-induced inflammatory response. In addition, the present invention provides a method of reducing the symptoms characteristic of an autoimmune disease by downregulating interleukin-12 production, comprising administering to the subject an amount of a ligand of complement receptor 3 or complement receptor 4 effective in downregulating interleukin-12 production, thereby reducing the symptoms characteristic of an autoimmune disease. Further provided is a method of treating or preventing the interleukin-12-induced inflammatory response of an autoimmune disease in a human subject, comprising administering to a subject an amount of a ligand of complement receptor 3 or complement receptor 4 effective in downregulating production of interleukin-12, thereby treating or preventing the interleukin-12-induced inflammatory response of an autoimmune disease.

This application is a continuation of U.S. application Ser. No. 09/196,867, filed Nov. 20, 1998, which claims priority to U.S. provisional application Serial No. 60/066,238, filed November, 1997, and these applications are herein incorporated by these references in their entirety.

### BACKGROUND OF THE INVENTION

1. Field of the Invention

The present invention relates to a method of modulating an IL-12 induced inflammatory response in a subject via binding of ligand to complement receptors. In particular, the present invention provides a method for reducing an IL-12 induced inflammatory response and treating an autoimmune disease, comprising administering a ligand of complement receptor 3 (CR3) or a ligand of complement receptor 4 (CR4) to a subject which has the effect of downregulating interleukin-12 production, thereby reducing an IL-12 induced inflammatory response and treating an autoimmune disease.

2. Background Art

CR3 (CD11b/CD18) is a heterodimeric molecule which, like lymphocyte function-related antigen 1 (LFA-1, CD11a/CD18) and CR4 (CD11c/CD18), belongs to the β₂-integrin family of cell adhesion molecules (1-5). CR3 is mainly expressed on polymorphonuclear leukocytes, monocytes/macrophages, and natural killer cells (1-2), and interacts with a variety of substances including complement fragment iC3b, intercellular adhesion molecule 1 (ICAM-1), fibrinogen, and β-glucan (2,4). Moreover, it mediates the binding of opsonized or unopsonized infectious agents such as Histoplasma capsulatum, Leishmania major, group B streptococci, Bordetella pertussis, Candida albicans and several mycobacteria (2,5-7,9).

Prior studies have shown that CR3 is involved in several monocyte/macrophage functions including transmigrational adhesion (2,4,8), phagocytosis, nitric oxide production and the generation of a respiratory burst (2,3,5,8,10). In addition, CR3 signaling may indirectly affect T cell function, as indicated in studies in which the administration of antibodies to CR3 to animals suppressed delayed type hypersensitivity (DTH) reactions (11,12) and fatally potentiated infections with Listeria monocytogenes or Toxoplasma gondii (13,14).

CR4 (CD11c/CD18) is a heterodimeric molecule, which, like CR3 (CD11b/CD18) and LFA-1 (CD11a/CD18), is a member of the β₂-integrin family of adhesion molecules (1-5). It is expressed in a pattern very similar to that of CR3, i.e., on polymorphonuclear leukocytes, monocytes/macrophages, dendritic cells, NK cells and some B cells and cytotoxic T cells. It interacts with many of the same substances as CR3, e.g., iC3b, LPS, ICAM-1, fibrinogen and β-glucan. Binding of a ligand to CR4 results in enhanced phagocytosis and CR4 is thought to play a role in transmigrational adhesion and antibody-dependent cellular cytotoxicity (1-4).

Interleukin-12 is a recently characterized cytokine with unique structure and pleiotropic effects (36-39). It consists of two disulfide-linked subunits, p40 and p35, that form functionally active p40/p35 heterodimers or inhibitory p40 homodimers. IL-12 is produced mainly by macrophages/monocytes and can be efficiently induced by intracellular parasites, bacteria and bacterial products. Functional studies have shown that IL-12 enhances cytolytic activity of natural killer (NK) cells and macrophages and induces, in synergism with the B7/CD28 interaction, cytokine production and proliferation of activated NK cells and T cells (40). Furthermore, IL-12 plays a pivotal role in Th1 T cell differentiation and induces naive T cells to produce IFN-γ. As a result of this ability to drive T cell responses to the Th1 phenotype, administration of IL-12 has been shown to be an effective treatment of mice with established parasitic infections, which elicit a Th2 T cell response (41,42).

The present invention provides a novel approach to downregulating IL-12 production in an autoimmune disorder due to the discovery that IL-12 production can be modulated via the complement receptors CR3 and/or CR4. Accordingly, the present invention provides methods for treating autoimmune diseases and other disorders associated with IL-12 production and release, by the administration, to subject, of a ligand of CR3 or CR4 which has the effect of downregulating IL-12 production within cells expressing the respective receptor.

### SUMMARY OF THE INVENTION

The present invention provides a method of downregulating interleukin-12 production in a subject comprising administering to the subject an IL-12 downregulating amount of a ligand of CR3 or a ligand of CR 4 effective in downregulating IL-12 production.

In addition, the present invention provides a method of reducing an IL-12-induced inflammatory response in a subject, comprising administering to the subject an amount of a ligand of CR3 or a ligand of CR4 effective in reducing the IL-12-induced inflammatory response.

Further provided is a method of reducing the symptoms characteristic of an autoimmune disease by downregulating IL-12 production, comprising administering to the subject an amount of a ligand of CR3 or a ligand of CR4 effective in downregulating IL-12 production, thereby reducing the symptoms characteristic of an autoimmune disease.

A method of treating or preventing the IL-12-induced inflammatory response of an autoimmune disease in a subject is also provided, comprising administering to a subject an amount of a ligand of CR3 or a ligand of CR4 effective in downregulating production of interleukin-12, thereby treating or preventing the IL-12-induced inflammatory response of an autoimmune disease.

Further provided is a method of treating or preventing the IL-12-induced inflammatory response of an inflammatory bowel disease in a subject, comprising administering to a subject an amount of a ligand of CR3 or a ligand of CR4 effective in downregulating production of interleukin-12, thereby treating or preventing the IL-12-induced inflammatory response of an inflammatory bowel disease.

Also provided is a method of treating or preventing the IL-12-induced inflammatory response of septic shock in a subject, comprising administering to a subject a ligand of CR3 or a ligand of complement receptor 4 effective in downregulating production of interleukin-12, thereby treating or preventing the IL-12-induced inflammatory response of septic shock.

Finally, the present invention provides methods of screening a substance for the ability to downregulate IL-12 production upon binding CR3 or CR4, comprising: a) contacting the substance with cells that express CR3 or CR4 and produce IL-12; b) detecting a reduction of IL-12 production in the cells of step (a), whereby a reduction of IL-12 production indicates a substance having the potential to downregulate IL-12 production; and c) determining that the substance identified in step (b) downregulates IL-12 production by binding with CR3 or CR4 by competitive assays, thereby identifying a substance having the ability to downregulate interleukin-12 production upon binding CR3 or CR4.

### DETAILED DESCRIPTION OF THE PREFERRED EMBODIMENTS

The present invention may be understood more readily by reference to the following detailed description of specific embodiments and the Examples included herein. As used in the claims, “a” can include one or more.

The present invention is based on the surprising discovery that IL-12 production can be regulated via the binding of ligands to CR3 and/or CR4, identifying a new locus of control for T cell mediated immunity via these complement components. Accordingly, the present invention provides a method of downregulating IL-12 production in a subject, comprising administering to the subject an IL-12 downregulating amount of a ligand of CR3 and/or a ligand of CR4 effective in downregulating IL-12 production. The efficacy of a ligand of CR3 and/or CR4 in downregulating IL-12 production in a subject can be determined by measuring the amount of IL-12 in the subject according to assays for cytokine measurement which are commercially available, as well as the protocols described herein and any other suitable cytokine measuring assay.

The present invention also provides a method of reducing an IL-12-induced inflammatory response in a subject, comprising administering to the subject an amount of a ligand of CR3 and/or a ligand of CR4 effective in reducing the IL-12-induced inflammatory response. As used herein, “IL-12-induced inflammatory response” means any normal or abnormal immunologic response that in any fashion is dependent on IL-12.

The ligand of this invention can be, but is not limited to, any substance which has been shown by standard assays well known in the art and as described in the Examples provided herein, to bind either CR3 or CR4 (19) and to downregulate IL-12 production upon binding CR3 or CR4, respectively. Downregulation of IL-12 can be determined by measurement of IL-12 directly from cultures of cells stimulated to produce IL-12 in vitro, or directly in the serum of animals given a parenteral stimulus, such as LPS, or from animals or humans with inflammation. Assays to measure IL-12 directly include sandwich enzyme-linked immunosorbent assay (ELISA) or measurement of IL-12 bioactivity using a responsive cell line. Examples of the latter include the detection of interferon-γ (IFN-γ) production from IL-12 responsive T cell lines or from spleen cells from SCID mice. In addition IL-12 mRNA (for both the p40 and p35 subunits) can be measured by RT-PCR techniques from IL-12 producing cells, either following culture with CR3 and/or CR4 ligands, followed by more direct stimuli of IL-12, such as LPS and IFN-γ, or directly isolated from tissues (33).

In the present invention, the ligand for CR3 can be, but is not limited to, an antibody to CR3, iC3b, ICAM-1, fibrinogen, β-glucan, C3b, ICAM-2, ICAM-3, a CR3-binding microorganism and/or a product of a CR3-binding microorganism. Furthermore, the CR3-binding microorganism can be Histoplasma capsulatum, Cryptococcus neoformans, Mycobacteria species, group B streptococci, Leishmania species and Candida species.

Examples of ligands for CR4 include, but are not limited to antibodies to CR4, iC3b, ICAM-1, LPS, fibrinogen, β-glucan and CR4-binding microorganisms such as Histoplasma capsulatum and Cryptococcus neoformans, as well as any CR4-binding product of a microorganism that binds CR4.

It is to be understood that the ligand for CR3 or the ligand for CR4 of this invention can be a ligand (e.g., an antibody) which binds to CR3 or CR4 and enhances activation of binding of other ligands to CR3 or CR4. An example of such an antibody is monoclonal antibody M-M522, which specifically binds CD11b of the CR3 heterodimer. Thus, it is contemplated that all of the methods of the present invention as described herein can include one or more ligands of CR3 and/or one or more ligands of CR4.

Any animal which is subject to an IL-12 induced inflammatory response can be treated by this method although humans are the primary therapeutic target. Examples of nonhuman subjects that can be treated by the methods taught herein can include, but are not limited to, mice, rats, rabbits, dogs, cats, non-human primates, as well as any other species now known or later discovered to manifest an IL-12 induced inflammatory response.

An IL-12 induced inflammatory response in a subject is generally characterized by a variety of physical symptoms and manifestations including, but not limited to, fever, fatigue, weight loss, joint swelling, pain, tenderness, stiffness and skin lesions. In addition, an IL-12 induced inflammatory response can be identified by a clinical profile which includes an analysis of the results of various laboratory tests, such as erythrocyte sedimentation rate (ESR), complete blood count (CBC), serological assay for rheumatoid factor, examination of cerebrospinal fluid (CSF), blood chemistry analysis, cytokine (IL-12) measurement, as well as an analysis of X-rays, CT/MRI scans, tissue biopsies and the like. These symptoms and clinical parameters can be monitored in a subject to determine the efficacy of administration of the ligand of CR3 or CR4 in reducing the IL-12-induced inflammatory response.

The present invention also provides a method of reducing the symptoms characteristic of an autoimmune disease by downregulating IL-12 production, comprising administering to the subject an amount of a ligand of CR3 and/or a ligand of CR4 effective in downregulating IL-12 production, thereby reducing the symptoms characteristic of an autoimmune disease.

The symptoms characteristic of autoimmune disease include, but are note limited to, fever, fatigue, weight loss, joint swelling, pain, tenderness, stiffness, lymphadenopathy, visual acuity problems, abnormal blood glucose levels, pulmonary symptoms and skin lesions. The efficacy of administration of a ligand to CR3 and/or CR4 in downregulating IL-12 production in a subject can be determined by monitoring these and other symptoms manifested by a subject with an autoimmune disease. The lessening of severity or obliteration of one or more symptom indicates effective downregulating of IL-12 production in the subject.

The present invention additionally provides a method of treating or preventing an IL-12 induced inflammatory response in a subject comprising administering to the subject an amount of a ligand of CR3 and/or CR4 effective in treating the IL-12 induced inflammatory response.

The present invention further provides a method of treating or preventing the IL-12-induced inflammatory response of an autoimmune disease in a subject, comprising administering to a subject an amount of a ligand of CR3 and/or CR4 effective in downregulating production of interleukin-12, thereby treating or preventing the IL-12-induced inflammatory response of an autoimmune disease.

As used herein, autoimmune disease describes a disease state or syndrome whereby a subject's body produces a dysfunctional immune response against the subject's own body components, with adverse effects. This may include production of B cells which produce antibodies with specificity for all antigens, allergens or major histocompatibility (MHC) antigens or production of T cells bearing receptors recognizing self components and producing cytokines that cause inflammation. The autoimmune disease of the present invention can be, but is not limited to, inflammatory bowel disease (e.g., Crohn's disease, ulcerative colitis, celiac disease/tropical sprue), multiple sclerosis, rheumatoid arthritis, diabetes mellitus, pernicious anemia, autoimmune gastritis, psoriasis, Bechet's disease, idiopathic thrombocytopenic purpura, Wegener's granulomatosis, autoimmune thyroiditis, autoimmune oophoritis, bullous pemphigoid, pemphigus, polyendocrinopathies, Still's disease, Lambert-Eaton myasthenia syndrome, myasthenia gravis, Goodposture's syndrome, autoimmune orchitis, autoimmune uveitis, systemic lupus erythematosus, Sjogren's syndrome and ankylosing spondylitis.

In particular, it is contemplated that the present invention provides a method of treating or preventing the IL-12-induced inflammatory response of an inflammatory bowel disease (e.g., Crohn's disease, ulcerative colitis, celiac disease/tropical sprue) in a subject, comprising administering to a subject an amount of a ligand of CR3 and/or CR4 effective in downregulating production of interleukin-12, thereby treating or preventing the IL-12-induced inflammatory response of an inflammatory bowel disease.

The IL-12-induced inflammatory response of an autoimmune disease and in particular of an inflammatory bowel disease can be identified in a subject according to the symptoms and parameters of the subject's clinical profile, as described above. The efficacy of the administration of a ligand of CR3 and/or CR4 in downregulating production of IL-12, thereby treating the IL-12-induced inflammatory response of an autoimmune disease, can be determined by observing changes in the subject's symptoms and parameters of the subject's clinical profile, whereby an improvement in symptoms or change in one or more parameter from abnormal to normal or less abnormal indicates an effective ligand of CR3 and/or CR4.

The efficacy of the administration of a ligand of CR3 and/or CR4 in downregulating production of IL-12, thereby preventing the IL-12-induced inflammatory response of an autoimmune disease, can be determined by identifying a subject to be at risk of developing an autoimmune disease, administering the ligand of this invention to the subject at risk and monitoring the subject over time for the development of an IL-12 induced inflammatory response of an autoimmune disease according to the methods provided herein.

A method of treating or preventing the IL-12-induced inflammatory response of septic shock in a subject is also provided, comprising administering to a subject a ligand of CR3 and/or CR4 effective in downregulating production of interleukin-12, thereby treating or preventing the IL-12-induced inflammatory response of septic shock. As used herein, “septic shock” describes a disease state characterized by fever, low blood pressure, multiorgan system failure, disseminated intravascular coagulation (DIC). A diagnosis of septic shock can be further established by detection and monitoring of such clinical parameters as laboratory tests, including LDH, CRP and lactate, as well as arterial blood gas analysis.

The efficacy of treating an IL-12 induced inflammatory response of septic shock by administering a ligand of CR3 and/or CR4 can be determined by observing changes in the subject's symptoms and parameters of the subject's clinical profile, whereby an improvement in symptoms or change in one or more parameter from abnormal to normal or less abnormal indicates an effective ligand of CR3 and/or CR4.

The efficacy of the administration of a ligand of CR3 and/or CR4 in downregulating production of interleukin-12, thereby preventing the IL-12-induced inflammatory response of an septic shock, can be determined by identifying a subject to be at risk of developing septic shock, administering the ligand of this invention to the subject at risk and monitoring the subject over time for the development of an IL-12 induced inflammatory response of septic shock according to the methods provided herein. A subject can be identified as being at risk of developing septic shock according to the symptoms and clinical parameters provided herein and in particular, the development of fever, low blood pressure and signs and symptoms of early organ failure, as would be well known to a clinician.

As a further embodiment, the present invention provides a method of screening a substance for the ability to downregulate IL-12 production upon binding CR3, comprising: a) contacting the substance with cells that express CR3 and produce IL-12; b) detecting the presence or absence of IL-12 production in the cells of step (a), whereby an absence of IL-12 production indicates a substance having the potential to downregulate IL-12 production; and c) determining that the substance identified in step (b) downregulates IL-12 production by binding with CR3 by competitive binding assays, as are well known in the art, thereby identifying a substance having the ability to downregulate interleukin-12 production upon binding CR 3. For example, the competitive binding assay can comprise the steps of contacting the substance of step (b) and a known non-activating or inhibiting ligand of CR3 with the cells of step (a) and detecting inhibition of the ability of the substance of step (b) to downregulate IL-12 production, whereby inhibition of the ability of the substance of step (b) to downregulate IL-12 production identifies the substance of step (b) as a substance which binds CR3. The non-activating or inhibiting CR3 ligand of this assay can be, but is not limited to, non-activating or inhibiting antibodies to CR3 and soluble CR3 receptors, as well as any other substance now known or later identified to be a non-activating or inhibiting ligand of CR3.

Cell types which can be used in the above CR3 ligand binding screening method include, but are not limited to, human monocytes, neutrophils and myelomonocytic cell lines, such as THP-1 (ATCC TIB 202) or RPMI 1640 cells, as well as any other cell type now known or later identified to express CR3 and produce IL-12 which can be detected by the assays of this invention.

Also provided is a method of screening a substance for the ability to downregulate IL-12 production upon binding CR4, comprising: a) contacting the substance with cells that express CR4 and produce IL-12; b) detecting the presence or absence of IL-12 production in the cells of step (a), whereby an absence of IL-12 production indicates a substance having the potential to downregulate IL-12 production; and c) determining that the substance identified in step (b)downregulates IL-12 production by binding with CR4 by competitive binding assays, as are well known in the art thereby identifying a substance having the ability to downregulate interleukin-12 production upon binding CR4. For example, the competitive binding assay can comprise the steps of contacting the substance of step (b) and a known non-activating or inhibiting ligand of CR4 with the cells of step (a) and detecting inhibition of the ability of the substance of step (b) to downregulate IL-12 production, whereby inhibition of the ability of the substance of step (b) to downregulate IL-12 production identifies the substance of step (b) as a substance which binds CR4. The non-activating or inhibiting CR4 ligand of this assay can be, but is not limited to, non-activating or inhibiting antibodies to CR4 and soluble CR4 receptors, as well as any other substance now known or later identified to be a non-activating or inhibiting ligand of CR4.

Cell types which can be used in the above CR4 ligand binding screening method include, but are not limited to, human monocytes, neutrophils and myelomonocytic cell lines, such as THP-1 (ATCC TIB 202) or RPMI 1640 cells, as well as any other cell type now known or later identified to express CR4 and produce IL-12 which can be detected by the assays of this invention.

As described above, the ligand of this invention can be any substance that binds CR3 or CR4 and has the effect of downregulating IL-12 production in the cell to which it binds. For example, the ligand of this invention can be an antibody to CR3 or an antibody to CR4. The antibodies of this invention can be either monoclonal or polyclonal and can be from any source. For example, monoclonal or polyclonal antibodies can be produced which specifically bind either CR3 or CR4 and screened for specificity and activity according to protocols well known in the art (25). The antibody can bind both CR3 and CR4. However, to reduce the immunogenicity of the immunoglobulins themselves, antibodies are preferably of human origin or are antibodies generated in other species and “humanized” for administration in humans as described in the Examples provided herein. The antibodies of this invention can be fragments which retain the ability to bind their specific antigens.

The ligand of this invention can be administered orally or parenterally to the subject in a pharmaceutically acceptable carrier. Suitable carriers for oral administration of the antigen include one or more substances which may also act as flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pyrogen free saline, pharmaceutically accepted oils, or a mixture of any of these. The liquid can also contain other suitable pharmaceutical additions such as buffers, preservatives, flavoring agents, viscosity or osmo-regulators, stabilizers or suspending agents. Examples of suitable liquid carriers include water with or without various additives, including carboxypolymethylene as a pH-regulated gel. The ligand may be contained in enteric coated capsules that release the ligand into the intestine to avoid gastric breakdown.

For parenteral administration of the ligand, a sterile solution or suspension is prepared in saline that may contain additives, such as ethyl oleate or isopropyl myristate, and can be injected, for example, into subcutaneous or intramuscular tissues, as well as intravenously.

Alternatively, the ligand may be microencapsulated with either a natural or a synthetic polymer into microparticles 4-8 μm in diameter, which target intestinal lymphoid tissues and produce a sustained release of ligand for up to four weeks (43,44).

The ligand of this invention can be administered to the subject in amounts sufficient to downregulate IL-12 production, reduce an IL-12 induced inflammatory response and/or treat or prevent autoimmune disease. Optimal dosages used will vary according to the individual being treated and ligand being used. The amount of ligand will also vary among individuals on the basis of age, size, weight, condition, etc. One skilled in the art will realize that dosages are best optimized by the practicing physician and methods for determining dosage are described, for example, in Remington's Pharmaceutical Sciences (45).

Typically, for treatment of humans, ligand to CR3 and/or CR4 would be administered parenterally in a dosage ranging from 0.1 to 1000 mg/kg/body weight/day with a preferred dosage range of 1-10 mg/kg/day and most preferred dosage of 10-50 mg/kg/day. Ligands can be administered daily, up to three times a week for between one week and four months. Administration of the ligand can be stopped completely following a prolonged remission or stabilization of disease signs and symptoms and readministered following a worsening of either the signs or symptoms of the disease, or following a significant change in immune status, as determined by routine follow-up immunological studies well known to a clinician in this field (e.g., a return to significant reactivity of immune cells to a particular suspected or known disease-causing antigen or to a particular tolerogen (49-67).

The efficacy of administration of a particular dose of a ligand for CR3 and/or CR4 in reducing an IL-12 induced inflammatory response, downregulating IL-12 production, treating or preventing the IL-12 induced inflammatory response of an autoimmune disease and/or treating or preventing the IL-12 induced inflammatory response of septic shock can be determined by evaluating the particular aspects of the medical history, the signs, symptoms and objective laboratory tests that have a documented utility in evaluating inflammation and disease activity. These signs, symptoms and objective laboratory tests will vary depending on the particular disease being treated or prevented, as will be well known to any clinician in this field. For example, if, based on a comparison with an appropriate control group and knowledge of the normal progression of disease in the general population or the particular individual, 1) a subject's frequency or severity of recurrences is shown to be improved, 2) the progression of the disease is shown to be stabilized, or 3) the need for use of other immunosuppressive medications is lessened, then a particular treatment will be considered efficacious.

In a particular example, in using the ligands of the present invention to treat an autoimmune disease such as multiple sclerosis, clinical parameters and symptoms which can be monitored for efficacy can include the severity and number of attacks; or for continuously progressive disease, the worsening of symptoms and signs; the cumulative development of disability; the number or extent of brain lesions as determined by magnetic resonance imaging; and the use of immunosuppressive medications (49,50).

Once it is established that the IL-12 induced inflammatory response has been reduced and/or that disease activity is significantly improved or stabilized by a particular ligand treatment, specific signs, symptoms and laboratory tests will be evaluated in accordance with a reduced or discontinued treatment schedule. If an inflammatory response or disease activity recurs, based on standard methods of evaluation of the particular signs, symptoms and objective laboratory tests as described herein, treatment can be reinitiated.

Additionally, the efficacy of administration of a particular dose of a ligand of CR3 and/or CR4 in preventing an autoimmune disease in a subject not known to have an autoimmune disease, but known to be at risk of developing an autoimmune disease, can be determined by evaluating standard signs, symptoms and objective laboratory tests, known to one of skill in the art, over time. This time interval may be long (years/decades). The determination of who would be at risk for the development of an autoimmune disease would be made based on current knowledge of the known risk factors for a particular disease familiar to clinicians and researchers in this field, such as a particularly strong family history of disease.

The present invention is more particularly described in the following examples which are intended as illustrative only since numerous modifications and variations therein will be apparent to those skilled in the art.

### EXAMPLES

### Cell Isolation, Cell Culture Conditions and Assessment of Cytokine Production

Human monocytes were obtained from healthy donors (total n=14) by standard leukapheresis and were highly purified (95-99% purity) by counterflow centrifugal centrifugation (15). Cell purity was checked by flow cytometry analysis using monoclonal antibodies to CD14 and CD11b (Becton Dickinson, San Jose, Calif.). Monocytes were cultured at 2×10⁶ cells/ml for 24 h in 1 ml of RPMI 1640 medium (Whittaker, Walkersville, Md.) supplemented with 10% fetal calf serum (Whittaker), 100 μg/ml penicillin, 100 μg/ml streptomycin and 0.03% glutamine and stimulated at the beginning of the culture with the following substances as indicated: heat-killed, formalin-fixed Staphylococcus aureus cells (SAC; Cowan I strain, Calbiochem, Cambridge, Mass.), lipopolysaccharides (LPS) (Escherichia coli serotype 0127:B8, Sigma, St. Louis, Mo.); (the latter two substances bind via CD14) (16,17); recombinant interferon-γ (IFN-γ) (Genzyme, Cambridge, Mass.), CD40L trimer (which binds to CD40; Immunex Corporation, Seattle, Wash.), recombinant human soluble ICAM-1 (R+D System, Minneapolis, Minn.), interferon-α (IFN-α) (Endogen, Cambridge, Mass.), or anti-IFN-γ (Endogen). One experiment was typically performed with the monocytes from one donor and there was variability in individual amounts of IL-12 production (e. g., range for SAC plus IFN-γ stimulation: 380-3300 pg/ml IL-12 p70, mean approx. 1750 pg/ml). Monocytes were incubated with heat-killed HC (strain GS-57; Dr. R. Seder, Lymphokine Regulation Unit, NIAID, NIH) or iC3b-SRBC (2×10⁷/ml) 2 h prior to stimulation as indicated. iC3b-SRBC were prepared by sequential addition of anti-sheep erythrocytes (IgM; Pharmingen, San Diego, Calif.) and C5-deficient mouse serum (Sigma) to SRBC as described (18,19). The presence of iC3b on the cell surface was verified by flow cytometry showing a >10 fold increase in fluorescence intensity of staining with a monoclonal antibody to the iC3b neoantigen (Quidel, San Diego, Calif.). Human PBMC were isolated by Ficoll-Hypaque density gradient centrifugation from leukocyte concentrates prepared by automated leukapheresis of healthy donors (total n=5) as described (15). PBMC were cultured similar to monocytes, except that they were cultured at 1×10⁶ cells/ml for 48 h and stimulated with PHA (Sigma). In some experiments, recombinant human IL-12, recombinant IL-2, or antibodies to IL-12 (clone C17.8; all reagents from Pharmingen) were added as indicated.

Culture supernatants were assessed in duplicates by enzyme linked immunosorbent assay (ELISA) using kits or antibody pairs for IL-12 p70, TNF-α (both from R+D Systems), IL-12 p40, IL-10 (both from Pharmingen), IL-6, IL-1β, IFN-α, IFN-γ, (all from Endogen, Cambridge, Mass.) and TGF-β (Genzyme). Unstimulated cultures (with or without added integrin antibodies) contained cytokines below the detection limit (IL-12 p70>5 pg/ml, IL-12 p40>20 pg/ml, IFN-α>10 pg/ml, IL-10>100 pg/ml, TGF-β>50 pg/ml, IL-6>50 pg/ml, IL-1β>25 pg/ml). For TNF-α, the detecting limit was >25 pg/ml and the background was below 200 pg/ml. Integrin antibodies were added at the beginning of the culture in the indicated concentrations and were obtained in a preservative free preparation or dialyzed overnight before use. The antibodies and monocyte stimulants (e. g. CD40L trimer, ICAM-1) contained low endotoxin levels (as per information by the manufacturer or as demonstrated in appropriate assays performed in the presence of 1-5 μg/ml of polymyxin B (Sigma), which revealed little or no effect on IL-12 and TNF-α production). Antibodies were bound to plastic culture plates by overnight incubation with 100 μl of carbonate buffer at 4° C. overnight followed by washing with PBS and bound to polystyrene beads (goat anti-mouse IgG, Dynabeads M450, Dynal, Lake Success, N.Y.) according to the manufacturer's instruction. L-cells stably transfected with a plasmid expressing the FcγRII receptor (CD32w) as previously described (20,21) were incubated with 10 μl/ml of antibody at 37° C. for 1 hr, washed and used for the assays. Integrin antibodies were obtained from the American Type Culture Collection (ATCC, Rockville, Md.; clone LM2/1 (18), isotype: murine IgG1; and clone M1/70, rat anti mouse/human IgG2b), Bender MedSystems (Vienna, Austria; clone LM2/1), Monosan (Amsterdam, Netherlands; clone MEM-48, murine IgG1), Pharmingen (clones G43-25B [murine IgG2b], B-ly6 [murine IgG1], 44 [murine IgG1], 107.3 [murine IgG1], G555-178 [murine IgG2a]), Becton Dickinson (clone D12, murine IgG2a), and Caltag (San Francisco, Calif.; clone CLB-LFA-1/1, murine IgG1).

Western Blotting

Human monocytes (2.5×10⁷ in 5 ml per condition) were either untreated, treated with anti-CR3 (clone LM2/1, 10 μg/ml) for 20 min, treated with recombinant IFN-γ (1 μg/ml) for 5 min, or treated with LM2/1 for 20 min followed by IFN-γ for 5 min. Cells were then solubilized in a buffer consisting of 1% Triton X (Pierce, Rockford, Ill.), 150 mM NaCl, 50 mM Tris pH 8.0, 50 mM Na-pyrophosphate, 2.5 mM aprotinin, 2.5 mM leupeptin, 1 mM phenylmethylsulfonyl fluoride, and 1 mM NaVO₃ (all from Sigma). Protein concentration of the cell lysates was estimated by measuring the optical density at 280 nm based on the assumption that optical density of 1.4 corresponds to 1 mg/ml. Cell lysates were analyzed using Tris-glycine gels (Novex, San Diego, Calif.) with equivalent amounts of protein in each lane, followed by electrophoretic transfer to nitrocellulose membranes (Schleicher and Schuell, Keene, NH). After overnight blocking in 5% non-fat dry milk, 150 mM NaCl, 50 mM Tris and 0.05% Tween 20, membranes were incubated with horseradish peroxidase (HRP)-conjugated phosphotyrosine antibodies (Santa Cruz Biotechnology, Santa Cruz, Calif.) for 2 h. Membranes were then washed and developed with enhanced chemiluminescence (ECL; Pierce). Cell lysates from epidermal growth factor-stimulated A431 cells (Upstate Biotechnology Incorporation, Lake Placid, N.Y.) served as positive control for phosphotyrosine and STAT1 (signal transducers and activators of transcription) expression. For re-probing, membranes were stripped in 0.2 M glycine buffer (pH 2.8) for 30 min, washed, exposed to a rabbit polyclonal antibodies specifically reacting with STAT1 (Santa Cruz), washed again, incubated with a donkey anti-rabbit HRP conjugated antibody (Amersham Life Sciences, Arlington Heights, Ill.), and developed with enhanced chemiluminescence.

Treatment of Animals, LPS Challenge and Detection of Cytokine Levels

6-10 week old BALB/c mice housed under standard conditions were treated intraperitoneally with 0.5 ml of PBS containing either rat Ig (1 mg per mouse, Sigma), antibodies to CR3 (1 mg of clone M1/70 or 0.5 mg of 5C6 [Biosource International, Camarillo, Calif.]), or anti-IL-12 (1 mg, clone C17.8; Dr. G. Trinchieri, The Wistar Institute, Philadelphia, Pa.). This treatment was followed one hour later by a intravenous (tail vein) injection of LPS (1 μg per mouse in 100 μl of PBS) or PBS alone. Mice were then either sacrificed either three (for determination of IL-12 p40) or six hours (for IL-12 p70 and IFN-γ) later by cervical dislocation after blood had been obtained by cardiac puncture from anesthetized mice. Serum was obtained after 30 min clotting of the blood at 37° C. and subsequent centrifugation. Serum levels of IL-12 (p40 and p70) and IFN-γ were assessed by ELISA using antibody pairs from Pharmingen. Injection of higher doses of anti-CR3 (e.g. 1 or 3 mg M1/70 per mouse at 12 h and/or 1 h prior to LPS challenge) had no additional suppressive effects on IL-12 or IFN-γ secretion. IL-12 p70 and IFN-γ were not detectable in mice which had received only PBS and were very low or undetectable at three hours after LPS administration or in anti-IL-12 treated mice.

Statistics. Statistical significance of differences was determined by Student's t-test where indicated.

It is understood that the protocols described herein for identifying and characterizing the IL-12 downregulating effects of ligands of CR3 can also be applied to ligands of CR4.

In initial studies it was determined whether antibodies to CR3 affect the secretion of IL-12 by highly purified human monocytes stimulated with Staphylococcus aureus cells (SAC) and interferon-γ (IFN-γ). Exposure of monocytes to monoclonal antibodies that bind to the functionally important I-domain of CR3 (CD11b; clone LM2/1, (18)) or to CD18 results in a dose dependent and profound reduction of IL-12 (p70 heterodimer and p40 monomer) secretion, whereas antibodies to CD11a had no effects. Similarly, with the use of CR4 (CD11c) antibodies, there were downregulating effects on IL-12 production. These cytokine protein levels correlated with downregulated messenger RNA levels for IL-12 p35 and p40 as determined by reverse-transcriptase PCR. The blockade of IL-12 production was observed with a variety of monocyte stimulants known to induce IL-12 (e. g., IFN-γ in combination with lipopolysaccharide [LPS], SAC or CD40L trimer) and with a panel of monoclonal antibodies to CR3 and CR4 (Table 1). In contrast, secretion of other monocyte products such as tumor necrosis factor α (TNF-α), IL-10, transforming growth factor β (TGF-β), IL-6 and IL-1β was not significantly altered by any of the above antibodies (as determined in the same cultures), nor was cell viability (trypan blue exclusion) or cell surface expression of CD14 (flow cytometry). The one exception was an anti-CR3-induced reduction of IFN-α secretion (Table 1), a macrophage-derived cytokine previously shown to enhance IL-12 induced Th1 development (22). For higher doses of CD11b abs (25 μg/ml), a further downregulation of IL-12 (<5 pg/ml) was observed, as well as a twofold increase in IL-6 (5450 pg/ml), a cytokine dichotomy previously described in individuals with human immunodeficiency virus (HIV) infection (23).

The ability of natural CR3 ligands to suppress IL-12 production was examined by using iC3b coated sheep red blood cells (iC3b-SRBC) and heat-killed Histoplasma capsulatum (HC), an organism that binds to CR3 (3,24). Similar to the findings with anti-CD11b, when human monocytes were incubated with iC3b-SRBC or heat-killed HC and subsequently stimulated with IFN-γ and either SAC, LPS, or CD40L trimer, a downregulation of IL-12 p70 production was observed, whereas TNF-α and IL-10 levels were largely unaffected. The inhibition of IL-12 production by HC was: 1) not due to direct suppression by IL-10, since the addition of anti-IL-10 antibodies (10 μg/ml) to the cultures only slightly increased the levels of IL-12; and 2) was related to the ability of HC to bind to β2 integrins, since the addition of a CD18 antibody (clone CLB LFA1/1 which did not directly inhibit IL-12 production, see Table 1) to these cultures partially reversed the suppressive effect of HC (threefold increase in IL-12 p70, i. e., to 40% of levels without HC).

The suppression of IL-12 production by antibodies and ligands to CR3 could be due either to the transmission of a direct inhibitory signal through the CR3 molecule, or to the blocking of a positive signal for IL-12 production, as could be provided through interactions between CR3 and ICAM-1 on neighboring monocytes. To clarify this issue, a number of studies were conducted, which together suggested that important cell-cell interactions, especially via ICAM-I/CR3, are not particularly important for the induction of IL-12. These studies thus indirectly supported the conclusion that CR3 antibodies act via a direct inhibitory signal. First, the relative production of IL-12 per monocyte following SAC and IFN-γ stimulation was not altered when cells were diluted over large ranges (≧1:1,000) prior to culture, i.e., resulting in decreased homotypic interactions. Second, the addition of blocking anti-ICAM-1 to monocyte cultures to block ICAM-1/CR3 interactions on neighboring monocytes did not suppress IL-12 production. And third, antibodies to CD11b (clone LM2/1) immobilized onto plastic culture plates, polystyrene beads coated with anti-mouse IgG (Dynal, Lake Success, N.Y.), or to FcγRII receptor (CD32)-expressing L cells, completely inhibited SAC plus IFN-γ induced IL-12 production, suggesting that the membrane-fixed subset of CR3 (i.e., which remains unbound to the solid supports (26)) is not capable of providing a positive signal for IL-12 production through an interaction with ICAM-1 on neighboring monocytes.

Because these studies provided only indirect evidence that anti-CR3 was acting to transmit a negative signal to monocytes, studies were conducted in order to look more directly for intracellular targets of inhibitory signaling by CR3. Inhibition of tyrosine phosphorylation by CR3 signaling seemed possible since this effect had been previously demonstrated in a system that involves a CR3 ligand. Thus, it has been shown that tyrosine phosphorylation in response to IFN-γ was suppressed in macrophages infected with L. donovani (27), an organism binding to monocyte CR3 by either gp63 or lipophosphoglycan (LPG) (3,7). Furthermore, a similar phenomenon has been described in human monocytes after the binding of immune complexes (28) and a role for β1-integrin engagement in tyrosine dephosphorylation has been established (29). Thus, the possibility that reduced production of IL-12 and IFN-α could follow a CR3-induced inhibition of IFN-γ signal transduction was examined. Incubation of monocytes with IFN-γ alone induced tyrosine phosphorylation of several proteins, one of which was identified as STAT1 (signal transducers and activators of transcription 1) by Western blotting with anti-STAT1 antibodies and by use of a positive tyrosine phosphorylated STAT1 control. Following preincubation with anti-CR3, IFN-γ-induced tyrosine phosphorylation of several proteins, including STAT1 was not seen, suggesting an inhibition of tyrosine phosphorylation, or alternatively, an accelerated dephosphorylation of tyrosine residues by an as yet unidentified protein tyrosine phosphatase.

The inhibition of IFN-γ-mediated STAT1 activation (i. e., phosphorylation) presents a potential mechanism by which signaling through CR3 could decrease monocyte IL-12 production, since there are two potential STAT1 binding sites (IFN-γ activation sequence (GAS) elements) in the human IL-12 p40 promotor (positions-127 to -119 and -277 to -268 of the published sequence) and since deletion mutations including these sites reduced transcription of a reporter gene (30). In addition, it is possible that the JAK-STAT-GAS signal transduction pathway may be important for the regulation of human IL-12 p35 gene transcription and translation which is induced by stimulation with IFN-γ (30), or be involved in the expression of gene products that are indirectly responsible for the upregulation of IL-12p35 or IL-12p40 gene transcription or translation. Finally, the possibility that in addition to its effects on IFN-γ signaling, anti-CR3 suppressed IL-12 production through an IFN-γ independent mechanism as well, was supported by the observation that the low level of IL-12 induced by SAC alone, which was unchanged with the addition of anti-IFN-γ, was also inhibited with anti-CR3.

Studies on functional effects of CR3 signaling were extended to a murine model of IL-12-dependent septic shock. In this model, the intravenous injection of LPS results in symptoms of septic shock as well as high serum levels of IFN-γ and IL-12, all of which can be inhibited by the systemic administration of anti-IL-12 (31). This in vivo model was selected to study the effects of the antibodies to CR3 because, when compared to others, this model should be much less dependent on cell trafficking. This is because the injected LPS should be rapidly delivered to all lymphoid tissues and the response to LPS is unlikely to depend on significant migration of cells, at least within the first 3 or 6 hours, the times at which IL-12 and IFN-γ, respectively, were measured.

BALB/c mice were given intraperitoneal injections of either CR3 antibodies (1 mg of clone M1/70 or 0.5 mg of 5C6, both of which are non-opsonizing antibodies (8,32)) or control IgG 1 hour prior to LPS injection and were sacrificed six hours later, at which time serum was obtained. Pretreatment with anti-CR3 reduced the serum levels of IL-12 (p40 and p70) by more than 4 and 2.5-fold, respectively, and similarly diminished the levels of IFN-γ 4-fold. The effects on IL-12 by treatment with M1/70 or 5C6 abs were, consistent with prior studies of these antibodies (8,13), not due to elimination of circulating leukocytes since total and differential white blood cell counts were similar in all treatment groups. In addition, whole spleen cells from anti-CR3-treated and LPS challenged mice stimulated in vitro with anti-CD3/anti-CD28 or PHA manifest reduced (2-4-fold) IFN-γ production when compared to control mice. In accordance with these findings, addition of anti-CD11b antibodies (10 μl/ml) markedly inhibited IFN-γ production in human monocyte cultures stimulated with either phytohemagglutinin (PHA) (3,310 pg/ml IFN-γ for isotype control, 1,100 pg/ml for anti-CD11b (LM2/1)), platebound anti-CD3 plus anti-CD28 (3250 pg/ml IFN-γ for isotype control, <10 pg/ml for anti-CD11b) or anti-CD2 plus anti-CD28 (2,450 pg/ml IFN-γ for isotype control, <10 pg/ml for anti-CD11b), but had no significant effect on cell viability or on cell proliferation (as determined by ³H-thymidine incorporation at 72 h). The above effects of anti-CR3 on IFN-γ production were reversed by addition of recombinant human IL-12 (1,100 pg/ml IFN-γ after PHA stimulation with anti-CR3, 3,210 pg/ml after addition of 20 ng/ml of IL-12) but not by IL-2, indicating the specificity for IL-12.

The effects of anti-CR3 antibodies in reducing the inflammatory response were also evaluated in another model of chronic inflammation, namely the trinitrochlorobenzene sulfonic acid (TNBS)-induced colitis model. Induction of a chronic granulomatous colitis was achieved by intrarectal administration of the haptenizing agent TNBS (2 mg in 50% ethanol in a total volume of 100 μl) intrarectally into SJL/J mice on day 0. Control mice were treated with 50% ethanol alone. On day 21, mice were given rat Ig (1 mg), anti-CR3 antibodies (1 mg) or anti-IL-12 antibodies (2 mg). Weight of mice was recorded as a read-out for the clinical condition on days 0, 21, 24 and 28, with five mice per group. Mice treated with anti-CR3 or with anti-IL-12 exhibited a rapid increase in body weight as compared to TNBS plus rat Ig-treated mice. Mice were sacrificed on day 28 and cells from the lamina propria were isolated by standard procedures. Following in vitro restimulation with anti-CD3/anti-CD28, the IFN-γ secretion of the cells was determined by ELISA. IFN-γ production in the TNBS group was abrogated following treatment with anti-CR3 or anti-IL-12.

To summarize the TNBS-induced colitis model data, the treatment of TNBS colitis, a Th1-mediated inflammatory disorder, with anti-CR3 antibodies resulted in a significant clinical improvement, as measured by increase of body weight as well as an abrogation of the Th1 cytokine (IFN-γ) response.

These data show that the IL-12 response, crucial for the initiation of most CMI functions (33), may be regulated by CR3 and/or CR4 signaling. Thus, they provide insight into mechanisms underlying the ability of CR3 antibodies to abolish DTH reactions (8,11,12), to ameliorate Th1 cell-mediated autoimmune diseases including antigen-induced arthritis (34) and experimental allergic encephalomyelitis (35), and to enhance the severity of infections with L. monocytogenes (13) or T. gondii (14). In addition, our studies demonstrating the specific inhibition of IL-12 by HC and iC3b-SRBC may explain the impaired CMI accompanying infections with CR3 binding microorganisms (e. g., L. major, mycobacteria, HIV) (3,68). In this regard they may provide a cogent reason for the diminished IL-12 production by mononuclear cells in HIV infected individuals (17) and in leishmaniasis (68). It is possible that CR3-induced suppression of IL-12 responses to the above microorganisms may result in suppressed monocytic nitric oxide production and respiratory burst, thereby explaining their ability to thrive in intracellular compartments.

Thus, in disease states as well as in normal responses to invading microorganisms, signaling through CR3 and/or CR4 may play an important role in regulating Th1-Th2 homeostasis via IL-12. This is important for understanding the pathogenesis of infectious diseases and may provide a new approach for therapeutic immunointervention targeting IL-12.

Administration of ligand to CR3 and/or CR4 to a human subject having an IL-12 induced inflammatory response. To reduce an IL-12 induced inflammatory response, to downregulate IL-12 production in a human subject and/or to treat or prevent an IL-12 induced inflammatory response of an autoimmune disease or septic shock in a human subject, 0.1-1000 mg/kg of ligand to CR3 and/or CR4 can be administered parenterally to the subject as often as daily, up to about three times a week, for between about one week and about four months, or until clinical parameters, i.e, signs, symptoms and objective laboratory tests with which clinicians in this field will be familiar, indicate reduction of inflammation and/or prolonged remission, stabilization or improvement.

Production of humanized mouse antibodies to CR3 and CR4. Rodent monoclonal or polyclonal antibodies can be modified according to the protocols set forth in Junghans et al. (46), Brown et al. (47) and Kettleborough et al. (48). Specifically, rodent antibodies can be modified for human administration by constructing, through recombinant DNA protocols known to one of skill in the art, a chimeric rodent-human antibody composed of rodent variable regions and human heavy and light chain constant regions. Another approach to humanizing rodent antibodies is to graft rodent complementarity-determining regions (CDRs) from the rodent variable regions into human variable regions. By using either of these approaches, rodent antibodies can be humanized for administration into human subjects.

Throughout this application, various publications are referenced. The disclosures of these publications in their entireties are hereby incorporated by reference into this application in order to more fully describe the state of the art to which this invention pertains.

Although the present invention has been described with reference to specific details of certain embodiments thereof, it is not intended that such details should be regarded as limitations upon the scope of the invention except as and to the extent that they are included in the accompanying claims.

### References

1. Springer, T. A. 1990. Adhesion receptors of the immune system. Nature 346: 425-434.

2. Patarroyo, M. 1989. Leukocyte adhesion to cells. Molecular basis, physiological relevance, and abnormalities. Scand. J. Immunol. 30: 129-164.

3. Cooper, N. R. 1991. Complement evasion strategies of microorganisms. Immunol. Today 12: 327-331.

4. Kishimoto, T. K., R. S. Larson, A. L. Corbi, M. L. Dustin, D. E. Staunton, and T. A. Springer. 1989. The leukocyte integrins. Adv. Immunol. 46:149-182.

5. Ross, G. D., and V. Vetvicka. 1993. CR3 (CD11b, CD18): a phagocyte and NK cell membrane receptor with multiple ligand specificities and functions. Clin. Exp. Immunol. 92: 181-184.

6. Van Strijp, J. A. G., D. G. Russell, E. Tuomanen, E. J. Brown, and S. D. Wright. 1993. Ligand specificity of purified complement receptor type three (CD11b/CD18, αₘβ₂, Mac-1): Indirect effects of an ARG-GLY-ASP (RGD) sequence. J. Immunol. 151:33243336.

7. Antal, J. M., J. V. Cunningham, and K. J. Goodrum. 1992. Opsonin-dependent phagocytosis of group B streptococci: Role of complement receptor type 3. Infect. Immun. 60:1114-1121.

8. Rosen, H. 1990. Role of CR3 in induced myelomonocytic recruitment: Insights from in vivo monoclonal antibody studies in the mouse. J. Leuk. Biol. 48: 465-469.

9. Goodrum, K. J., L. L. McCormick, and B. Schneider. 1994. Group B streptococcus nitric oxide production in murine macrophages is CR3 (CD11b/CD18) dependent. Infect. Immun. 62:3102-3107.

10. Anderson, D. C., and T. A. Springer. 1987. Leukocyte adhesion deficiency: An inherited defect in the Mac-1, LFA-1, and p150,95 glycoproteins. Annu. Rev. Med. 38:175-194.

11. Rosen, H., G. Milon, and S. Gordon. 1989. Antibody to the murine type 3 complement receptor inhibits T lymphocyte-dependent recruitment of myelomonocytic cells in vivo. J. Exp. Med. 169:535-549.

12. Patarroyo, M. 1994. Adhesion molecules mediating recruitment of monocytes to inflamed tissues. Immunobiol. 191:474-477.

13. Rosen, H., S. Gordon, and R. J. North. 1989. Exacerbation of murine listeriosis by a monoclonal antibody specific for the type 3 complement receptor of myelomonocytic cells. J. Exp. Med. 170:27-38.

14. Johnson, L. L., G. W. Gibson, and P. C. Sayles. 1996. CR3-dependent resistance to acute Toxoplasma gondii infection in mice. Infect. Immun. 64: 1998-2003.

15. Abrahamsen, T. G., C. S. Carter, E. J. Read, M. Rubin, H. G. Goetzman, E. F. Lizzio, Y. L. Lee, M. Hanson, P. A. Pizzo, T. Hoffman. 1991. Stimulatory effect of counterflow centrifugal elutriation in large-scale separation of peripheral blood monocytes can be reversed by storing cells at 37° C. J. Clin. Apheresis 6:48-53.

16. Zarewych, D. M., A. L. Kindzelskii, R. F. Todd III, and H. R. Petty. 1996. LPS induces CD14 association with complement receptor type three, which is reversed by neutrophil adhesion. J. Immunol. 156:430-433.

17. Kusunoki, T, E. Hailman, T. S.-C. Juan, H. S. Lichtenstein, and S. D. Wright. 1995. Molecules from Staphylococcus aureus that bind CD14 and stimulate innate immune responses. J. Exp. Med. 182:1673-1682.

18. Diamond, M. S., J. Garcia-Aguilar, J. K. Bickford, A. L. Corbi, and T. A. Springer. 1993. The I domain is a major recognition site on the leukocyte integrin Mac-1 (CD11b/CD18) for four distinct adhesion ligands. J. Cell Biol. 120:1031-1043.

19. Ross, G. D. 1980. Analysis of the different types of leukocyte membrane complement receptors and their interaction with the complement system. J. Immunol. Methods 37:197-211.

20. Kelsall, B. L., E. Stuber, M. F. Neurath, and W. Strober. 1996. Interleukin-12 production by dendritic cells: The role of CD40-CD40L interactions in Th1 T cell responses. Ann. N. Y. Acad. Sci. 795:116-126.

21. Peltz, G. A., M. L. Trounstein, and K. M Moore. 1988. Cloned and expressed human Fc-receptor for OgR mediated anti-CD3-dependent lymphoproliferation. J. Immunol. 141:1891-1896.

22. Wenner, C. A., M. L. Güler, S. E. Macatonia, A. O'Garra, and K. M. Murphy. 1996. Roles of IFN-γ and IFN-α in IL-12-induced T helper cell-1 development J. Immunol. 156:1442-1447.

23. Chemini, J., S. E. Starr, I. Frank, A. D'Andrea, X. Ma, R. R. MacGregor, J. Sennelier, and G. Trinchieri. 1994. Impaired interleukin-12 production in human immunodeficiency virus-infected patients. J. Exp. Med. 179:1361-1366.

24. Bullock, W. E., and S. D. Wright. 1987. Role of the adherence-promoting receptors, CR3, LFA-1, and p150,95, in binding of Histoplasma capsulatum by human macrophages. J. Exp. Med. 165:195-210.

25. Harlow and Lane, Antibodies; A Laboratory Manual, Cold Spring Harbor Laboratory, Cold Spring Harbor, New York, (1988).

26. Graham, I. L., H. D. Gresham, and E. J. Brown. 1989. An immobile subset of plasma membrane CD11b/CD18 (Mac-1) is involved in phagocytosis of targets recognized by multiple receptors. J Immunol. 142:2352-2358.

27. Nandan, D, and N. E. Reiner. 1995. Attenuation of gamma interferon-induced tyrosine phosphorylation in mononuclear phagocytes infected with Leishmania donovani: Selective inhibition of signaling through Janus kinases and Stat1. Infect. Immun. 63:4495-4500.

28. Feldman, G. M., E. J. Chuang, and D. S. Finbloom. 1995. IgG immune complexes inhibit IFN-γ-induced transcription of the FcγRI gene in human monocytes by preventing the tyrosine phosphorylation of the p91 (Stat1) transcription factor. J. Immunol. 154:318-325.

29. Lu, T. T., L. G. Yan, and J. A. Madri. 1996. Integrin engagement mediates tyrosine dephosphorylation on platelet-endothelial cell adhesion molecule 1. Proc. Natl. Acad. Sci. USA 93:11808-11813.

30. Ma, X., J. M. Chow, G. Gri, G. Carra, F. Gerosa, S. F. Wolf, R. Dzialo, and G. Trinchieri. 1996. The interleukin-12 p40 gene promoter is primed by interferon-γ in monocytic cells. J. Exp. Med. 183:147-157.

31. Wysocka, M., M. Kubin, L. Q. Vieira, L. Ozmen, G. Garotta, P. Scott, and G. Trinchieri 1995. Interleukin-12 is required for interferon-y production and lethality in lipopolysaccharide-induced shock in mice. Eur. J. Immunol. 25:672-676.

32. Springer, T., G. Galfre, D. S. Secher, and C. Milstein. 1978. Monoclonal xenogeneic antibodies to murine cell surface antigens: identification of novel leukocyte differentiation antigens. Eur. J. Immunol. 8:539-551.

33. Trinchieri, G. 1994. Interleukin-12: A cytokine produced by antigen-presenting cells with immunoregulatory functions in the generation of T-helper cells type 1 and cytotoxic lymphocytes. Blood 84:4008-4027.

34. Mazzone, A ., and G. Ricevuti. 1995. Leukocyte CD11/CD18 integrins: Biological and clinical relevance. Haematologica 80:161-175.

35. Gordon, E. J., K. J. Myers, J. P. Dougherty, H. Rosen, and Y. Ron. 1995. Both anti-CD11a (LFA-1) and anti-CD11b (Mac-1) therapy delay the onset and diminish the severity of experimental autoimmune encephalomyelitis. J. Neuroimmunol. 62:153-160.

36. Kobayashi, M., L. Fitz, M. Ryan, R. M. Hewick, S. C. Clark, S. Chan, R. Loudon, F. Sherman, B. Perussia, and G. Trinchieri. 1989. Identification and purification of natural killer cell stimulatory factor (NKSF), a cytokine with multiple biological effects on human lymphocytes. J. Exp. Med. 170:827-845.

37. Seder, R. A., R. Gazzinelli, A. Sher, and W. E. Paul. 1993. IL-12 acts directly on CD4+T cells to enhance priming for IFN-γ production and diminishes IL-4 inhibition of such priming. Proc. Natl. Acad. Sci. USA 90:10188-10192.

38. Ling, P., M. K. Gately, U. Gubler, A. S. Stern, P. Lin, K. Hollfelder, C. Su, Y.-C. E. Pan, and J. Hakimi. 1995. Human IL-12 p40 homodimer binds to the IL-12 receptor but does not mediate biologic activity. J. Immunol. 154:116-127.

39. Podlaski, F. J., V. B. Nanduri, J. D. Hulmes, Y.-C. E. Pan, W. Levin, W. Danho, R. Chizzonite, M. K. Gately, and A. S. Stern. 1992. Molecular characterization of interleukin 12. Arch. Biochem. Biophys. 294:230-237.

40. Kubin, M., M. Kamoun, and G. Trinchieri. 1994. Interleukin 12 synergizes with B7/CD28 interaction in inducing efficient proliferation and cytokine production of human T cells. J. Exp. Med. 180:211-222.

41. Wynn, T. A., I. Eltoum, I. P. Oswald, A. W. Cheever, and A. Sher. 1994. Endogenous interleukin 12 (IL-12) regulates granuloma formation induced by eggs of Schistosoma mansoni and exogenous IL-12 both inhibits and prophylactically immunizes against egg pathology. J. Exp. Med. 179:1551-1561.

42. Murray, H. W., and J. Hariprashad. 1995. Interleukin 12 is effective treatment for an established systemic intracellular infection: experimental visceral leishmaniasis. J. Exp. Med. 181:387-391.

43. Eldridge et al.. 1989. Cur. Topics in Microbiol. and Immunol., 146:59-65.

44. Oka et al. 1990. Vaccine, 8:573-576.

45. Martin, E. W. Remington's Pharmaceutical Sciences, Martin, latest edition, Mack Publishing Co., Easton, Pa.

46. Junghans et al. 1990. Cancer Research 50:1495-1502.

47. Brown et al. 1991. Proc. Natl. Acad. Sci. USA 88:2663-2667.

48. Kettleborough et al. 1991. Protein Engineering 4:773-783.

49. Van Oosten et al., 1995. Multiple sclerosis therapy. A practical guide. Drugs 49:200-12.

50. Rolak, L. A., 1996. The diagnosis of multiple sclerosis. Neurol. Clin. 14:27-43.

51. Mestecky et al., 1996. Induction of tolerance in humans: effectiveness of oral and nasal immunization routes. Ann N.Y. Acad. Sci. 778:194-201.

52. Husby et al., 1994. Oral tolerance in humans. T cell but not B cell tolerance after antigen feeding. J. Immunol. 152:4663-70.

53. Hohol et al., 1996. Three-year open protocol continuation study of oral tolerization with myelin antigens in multiple sclerosis and design of a phase III pivotal trial. Ann. N.Y. Acad. Sci.778:243-50.

54. Weiner et al., 1993. Double-blind pilot trial of oral tolerization with myelin antigens in multiple sclerosis. Science 259:1321-4.

55. Trentham et al., 1993. Effects of oral administration of type II collagen on rheumatoid arthritis. Science 261:1727-30.

56. Kardon, E. M., Acute asthma. Emerg. Med. Clin. North Am. 14:93-114.

57. Martin, R. J., 1994. Management of patients with chronic asthma: controversies and future directions. Ann. Allergy 72:390-2.

58. Powell, C. V., 1993. Management of acute asthma in childhood. Br. J. Hosp. Med. 50:272-5.

59. Lynch et al., 1996. Lung transplantation in chronic airflow limitation. Med. Clin. North Am. 80:657-70.

60. Corris, P. A., 1995. Post heart/lung transplantation management. J. R.. Soc. Med. 25:37-40.

61. Engeler, C. E., 1995. Heart-lung and lung transplantation. Radiol. Clin. North Am. 33:559-80.

62. Davis et al., 1995. Pulmonary transplantation. Ann. Surg. 221:14-28.

63. Kremer, J. M., 1995. The changing face of therapy for rheumatoid arthritis. Rheum. Dis. Clin. North Am. 21:845-52.

64. Dawes et al., 1995. Treatment of early rheumatoid arthritis: a review of current and future concepts and therapy. Clin. Exp. Rheumatol. 13:381-94.

65. Porter et al., 1993. Medical management of rheumatoid arthritis. B. M. J. 307:425-8.

66. Jaffe, I. A., 1992. New approaches to the management of rheumatoid arthritis. J. Rheumatol. 36:2-8.

67. Wilke et al., 1991. Therapy for rheumatoid arthritis: combinations of disease-modifying drugs and new paradigms of treatment. Semin. Arthritis Rheum. 21:21-34.

68. Carrera, L., R. T. Gazzinelli, R. Badolato, S. Hieny, W. Muller, R. Kuhn, and D. L. Sacks. 1996. Leishmania promastigotes selectively inhibit interleukin-12 induction in bone marrow-derived macrophages from susceptible and resistant mice. J. Exp. Med. 183:515-526.

TABLE 1
Effects of Integrin Antibodies on the Secretion of a Variety of Cytokines by Highly Purified Human Monocytes

| | | Isotype control | | | | | | CD18 | | | (CD4) |
|---|---|---|---|---|---|---|---|---|---|---|---|
| | | IgG1 | IgG2a | | CD11b | | | | (CLB- | CD11a | CD11c |
| | None | (clone 107.3) | (G555-178) | (LM2/1) | (44) | (M1/70) | (D12) | (MEM-48) | (LFA 1/1) | (G43-25B) | (B-ly6) |
| Cytokine | pg/ml | | | | | | | | | | |
| IL-12 (p70) | 2,180 | 2,040 | 2,155 | 260 | 735 | 780 | 670 | 185 | 1,720 | 1,625 | 770 |
| IL-12 (p40) | 23,450 | 21,960 | 19,870 | 2,400 | 18,170 | 13,140 | 19,350 | 3,280 | 21,580 | 22,200 | 12,410 |
| IFN-α | 250 | 265 | ND | 30 | 130 | 85 | 175 | 15 | 250 | 135 | 240 |
| TNF-α | 1,850 | 2,113 | 2,325 | 2,575 | 2,100 | 2,638 | 2,050 | 1,850 | 3,188 | 2,025 | 3,150 |
| IL-10 | 5,200 | 5,750 | 5,700 | 4,550 | 5,500 | 3,400 | 4,300 | 5,500 | 6,700 | 4,850 | 4,950 |
| TGF-β | 2,350 | 2,675 | 2,375 | 3,300 | 2,650 | 2,275 | 2,625 | 3,175 | 2,950 | 2,650 | 2,800 |
| IL-6 | 2,650 | 3,050 | ND | 4,275 | 3,700 | ND | 3,450 | 3,425 | 3,600 | 3,350 | 3,525 |
| IL-1β | 2,025 | 2,138 | ND | 2,050 | 2,025 | ND | 2,363 | 1,875 | 2,338 | 2,200 | 1,875 |
| Data represent antibody concentration of 10 μg/ml and were performed in a concentration range of 0.1-25 μg/ml. Data are means of duplicates from one experiment and are representative of three experiments. Percent suppression of IL-12 p70 in all three experiments was comparable, e.g., percent suppression of IL-12 p70 for clone LM2/1 ranged from 76-98%, for clone 44 (53-74%), for M1/70 (41-78%), for D12 | | | | | | | | | | | |
| #(48-85%), for MEM-48 (82-99%), for CLB LFA1/1 (12-22%), for G43 25B (−16-33%), and for B-ly6 (37-79%). The baseline of IL-12 p70 production varied in the three experiments (i.e., for three different donors) from 1,060 to 2,180 pg/ml. | | | | | | | | | | | |

ND denotes not determined.

What is claimed is:

1. A method of downregulating interleukin-12 production in a subject, comprising administering to the subject an interleukin-12 downregulating amount of a ligand of complement receptor 3 or complement receptor 4 effective in downregulating interleukin-12 production.

2. A method of reducing an interleukin-12-induced inflammatory response in a subject, comprising administering to the subject an amount of a ligand of complement receptor 3 or complement receptor 4 effective in reducing the interleukin-12-induced inflammatory response.

3. A method of reducing the symptoms characteristic of an autoimmune disease by downregulating interleukin-12 production, comprising administering to the subject an amount of a ligand of complement receptor 3 or complement receptor 4 effective in downregulating interleukin-12 production, thereby reducing the symptoms characteristic of an autoimmune disease.

4. The method of claim 3, wherein the symptoms characteristic of autoimmune disease are selected from the group consisting of fever, fatigue, weight loss, joint swelling, pain, tenderness, stiffness and skin lesions.

5. A method of treating or preventing the interleukin-12-induced inflammatory response of an autoimmune disease in a human subject, comprising administering to a subject an amount of a ligand of complement receptor 3 or complement receptor 4 effective in downregulating production of interleukin-12, thereby treating or preventing the interleukin-12-induced inflammatory response of an autoimmune disease.

6. The method of claim 5, wherein the autoimmune disease is selected from the group consisting of inflammatory bowel disease, multiple sclerosis, rheumatoid arthritis, diabetes mellitus, pernicious anemia, autoimmune gastritis, psoriasis, Bechet's disease, idiopathic thrombocytopenic purpura, Wegener's granulomatosis, autoimmune thyroiditis, autoimmune oophoritis, bullous pemphigoid, pemphigus, polyendocrinopathies, Still's disease, Lambert-Eaton myasthenia syndrome, myasthenia gravis, Goodposture's syndrome, autoimmune orchitis, autoimmune uveitis, systemic lupus erythematosus, Sjogren's syndrome and ankylosing spondylitis.

7. A method of treating or preventing the interleukin-12-induced inflammatory response of an inflammatory bowel disease in a human subject, comprising administering to a subject an amount of a ligand of complement receptor 3 or complement receptor 4 effective in downregulating production of interleukin-12, thereby treating or preventing the interleukin-12-induced inflammatory response of an inflammatory bowel disease.

8. The method of claim 7, wherein the inflammatory bowel disease is selected from the group consisting of Crohn's disease, ulcerative colitis and celiac disease/tropical sprue.

9. A method of treating or preventing the interleukin-12-induced inflammatory response of septic shock in a human subject, comprising administering to a subject an amount of a ligand of complement receptor 3 or complement receptor 4 effective in downregulating production of interleukin-12, thereby treating or preventing the interleukin-12-induced inflammatory response of septic shock.

10. The method of claim 1, 2, 3, 4, 5, 6, 7, 8 or 9, wherein the ligand of complement receptor 3 is selected from the group consisting of antibodies to complement receptor 3, iC3b, ICAM-1, fibrinogen, β-glucan, C3b, ICAM-2, ICAM-3, a complement receptor 3-binding microorganism and a complement receptor 3-binding product of a complement receptor 3-binding microorganism.

11. The method of claim 1, 2, 3, 4, 5, 6, 7, 8 or 9, wherein the ligand of complement receptor 4 is selected from the group consisting of antibodies to complement receptor 4, iC3b, I-Cam-1, LPS, fibrinogen, β-glucan, a complement receptor 4-binding microorganism and a complement receptor 4-binding product of a complement receptor 4-binding microorganism.

12. A method of screening a substance for the ability to downregulate interleukin-12 production upon binding complement receptor 3, comprising:
a) contacting the substance with cells that express complement receptor 3 and produce interleukin-12;
b) detecting a reduction of interleukin-12 production in the cells of step (a),whereby an reduction of interleukin-12 production indicates a substance having the potential to downregulate interleukin-12 production; and
c). determining that the substance identified in step (b) downregulates interleukin-12 production by binding with complement receptor 3 by competitive binding assays, thereby identifying a substance having the ability to downregulate interleukin-12 production upon binding complement receptor 3.

13. A method of screening a substance for the ability to downregulate interleukin-12 production upon binding complement receptor 4, comprising:
a) contacting the substance with cells that express complement receptor 4 and produce interleukin-12;
b) detecting the reduction of interleukin-12 production in the cells of step (a), whereby a reduction of interleukin-12 production indicates a substance having the potential to downregulate interleukin-12 production; and
c) determining that the substance identified in step (b) downregulates interleukin-12 production by binding with complement receptor 4 by competitive binding assays, thereby identifying a substance having the ability to downregulate interleukin-12 production upon binding complement receptor 4.
